(19) United States
(12) Patent Application Publication
(10) Pub. No.: US 2010/0267932 A1
(43) Pub. Date: Oct. 21, 2010

(54) PROCESS FOR THE PURIFICATION OF FC-FUSION PROTEINS

(76) Inventors: Alex Eon-Duval, Vevey (CH); Alain Lamproye, Bouloz (CH)

Correspondence Address: HOWREY LLP - East, C/O IP DOCKETING DEPARTMENT, 2941 FAIRVIEW PARK DR, SUITE 200, FALLS CHURCH, VA, 22042-2924, US

(21) Appl. No.: 12/377,119

(22) PCT Filed: Aug. 27, 2007
(86) PCT No.: PCT/EP07/58886

§ 371 Date: Jun. 25, 2010

Related U.S. Application Data
(60) Provisional application No. 60/842,682, filed on Sep. 6, 2006.

(30) Foreign Application Priority Data
Aug. 28, 2006 (EP) 06119610.1

(51) Int. Cl. C07K 1/18

(52) U.S. Cl. 530/387.3

(57) ABSTRACT

The invention relates to a process for the purification of an Fc-fusion protein having a pI between 6.9 and 9.5 comprising protein A or G affinity chromatography, cation exchange chromatography, anion exchange chromatography and hydroxyapatite chromatography.

### FIELD OF THE INVENTION

The present invention is in the field of protein purification. More specifically, it relates to the purification of Fc-fusion proteins via Protein A or Protein G affinity chromatography, cation exchange chromatography, anion exchange chromatography and hydroxyapatite chromatography.

### BACKGROUND OF THE INVENTION

Proteins have become commercially important as drugs that are generally called “biologicals”. One of the greatest challenges is the development of cost effective and efficient processes for purification of proteins on a commercial scale. While many methods are now available for large-scale production of proteins, crude products, such as cell culture supernatants, contain not only the desired product but also impurities, which are difficult to separate from the desired product. Although cell culture supernatants of cells expressing recombinant protein products may contain less impurities if the cells are grown in serum-free medium, the host cell proteins (HCPs) still remain to be eliminated during the purification process. Additionally, the health authorities request high standards of purity for proteins intended for human administration.

Many purification methods contain steps requiring application of low or high pH, high salt concentrations or other extreme conditions that may jeopardize the biological activity of a given protein. Thus, for any protein it is a challenge to establish a purification process allowing for sufficient purity while retaining the biological activity of the protein.

A number of chromatographic systems are known that are widely used for protein purification.

Ion exchange chromatography systems are used for separation of proteins primarily on the basis of differences in charge. In ion exchange chromatography, charged patches on the surface of the solute are attracted by opposite charges attached to a chromatography matrix, provided the ionic strength of the surrounding buffer is low. Elution is generally achieved by increasing the ionic strength (i.e. conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anion exchangers can be classified as either weak or strong. The charge group on a weak anion exchanger is a weak base, which becomes de-protonated and, therefore, looses its charge at high pH. DEAE-sepharose is an example of a weak anion exchanger, where the amino group can be positively charged below pH˜9 and gradually loses its charge at higher pH values. Diethylaminoethyl (DEAE) or diethyl-(2-hydroxy-propyl)aminoethyl (QAE) have chloride as counter ion, for instance. A strong anion exchanger, on the other hand, contains a strong base, which remains positively charged throughout the pH range normally used for ion exchange chromatography (pH 1-14). Q-sepharose (Q stands for quaternary ammonium) is an example for a strong anion exchanger.

Cation exchangers can also be classified as either weak or strong. A strong cation exchanger contains a strong acid (such as a sulfopropyl group) that remains charged from pH 1-14; whereas a weak cation exchanger contains a weak acid (such as a carboxymethyl group), which gradually loses its charge as the pH decreases below 4 or 5. Carboxymethyl (CM) and sulphopropyl (SP) have sodium as counter ion, for example.

A different chromatography resin is based on an insoluble hydroxylated calcium phosphate matrix called hydroxyapatite. Hydroxyapatite chromatography is a method of purifying proteins that utilizes an insoluble hydroxylated calcium phosphate (Ca₅(PO₄)₃OH)₂, which forms both the matrix and ligand. Functional groups consist of pairs of positively charged calcium ions (C-sites) and clusters of negatively charged phosphate groups (P-sites). The interactions between hydroxyapatite and proteins are complex and multi-mode. In one method of interaction, positively charged amino groups on proteins associate with the negatively charged P-sites and protein carboxyl groups interact by coordination complexation to C-sites (Shepard et al., 2000).

Crystalline hydroxyapatite was the first type of hydroxyapatite used in chromatography. Ceramic Hydroxyapatite (CHA) chromatography is a further development in hydroxyapatite chromatography. Ceramic hydroxyapatite has high durability, good protein binding capacity, and can be used at higher flow rates and pressures than crystalline hydroxyapatite. (Vola et al., 1993).

Hydroxyapatite has been used in the chromatographic separation of proteins, nucleic acids, as well as antibodies. In hydroxyapatite chromatography, the column is normally equilibrated, and the sample applied, in a low concentration of phosphate buffer and the adsorbed proteins are then eluted in a concentration gradient of phosphate buffer (Giovannini et al., 2000).

Yet a further way of purifying proteins is based on the affinity of a protein of interest to another protein that is immobilized to a chromatography resin. Examples for such immobilized ligands are the bacterial cell wall proteins Protein A and Protein G, having specificity to the Fc portion of certain immunoglobulins. Although both Protein A and Protein G have a strong affinity for IgG antibodies, they have varying affinities to other immunoglobulin classes and isotypes as well.

Protein A is a 43,000 Dalton protein that is produced by the bacteria Staphylococcus aureus and contains four binding sites to the Fc regions of IgG. Protein G is produced from group G Streptococci and has two binding sites for the IgG Fc region. Both proteins have been widely characterized for their affinity to various types of immunoglobulins. Protein L is a further bacterial protein, originating from Peptostreptococcus, binding to Immunoglobulins and fragments thereof containing Ig light chains (Akerstrom and Bjork, 1989).

Protein A, Protein G and Protein L affinity chromatography are widely used for isolation and purification of immunoglobulins.

Since the binding sites for Protein A and Protein G reside in the Fc region of an immunoglobulin, Protein A and Protein G affinity chromatography also allows purification of so-called Fc-fusion proteins.

Fc-fusion proteins are chimeric proteins consisting of the effector region of a protein, such as the binding region of a receptor, fused to the Fc region of an immunoglobulin that is frequently an immunoglobulin G (IgG). Fc-fusion proteins are widely used as therapeuticals as they offer advantages conferred by the Fc region, such as:
The possibility of purification using protein A or protein G affinity chromatography with affinities varying according to the IgG isotype. Human IgG₁, IgG₂ and IgG₄ bind strongly to Protein A and all human IgGs including IgG₃ bind strongly to Protein G;
An increased half-life in the circulatory system, since the Fc region binds to the salvage receptor FcRn which protects from lysosomal degradation;
Depending on the medical use of the Fc-fusion protein, the Fc effector functions may be desirable. Such effector functions include antibody-dependent cellular cytotoxicity (ADCC) through interactions with Fc receptors (FcγRs) and complement-dependent cytotoxicity (CDC) by binding to the complement component 1q (C1q). IgG isoforms exert different levels of effector functions. Human IgG₁ and IgG₃ have strong ADCC and CDC effects while human IgG₂ exerts weak ADCC and CDC effects. Human IgG₄ displays weak ADCC and no CDC effects.

Serum half-life and effector functions can be modulated by engineering the Fc region to increase or reduce its binding to FcRn, FcγRs and C1q respectively, depending on the therapeutic use intended for the Fc-fusion protein.

In ADCC, the Fc region of an antibody binds to Fc receptors (FcγRs) on the surface of immune effector cells such as natural killers and macrophages, leading to the phagocytosis or lysis of the targeted cells.

In CDC, the antibodies kill the targeted cells by triggering the complement cascade at the cell surface. IgG isoforms exert different levels of effector functions increasing in the order of IgG₄<IgG₂<IgG₁≦IgG₃. Human IgG₁ displays high ADCC and CDC, and is the most suitable for therapeutic use against pathogens and cancer cells.

Under certain circumstances, for example when depletion of the target cell is undesirable, abrogating or diminishing effector functions may be required. On the contrary, in the case of antibodies intended for oncology use, increasing effector functions may improve their therapeutic activity (Carter et al., 2006).

Modifying effector functions can be achieved by engineering the Fc region to either improve or reduce their binding to FcγRs or the complement factors.

The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Two regions of the CH2 domain are critical for FcγRs and complement C1q binding, and have unique sequences in IgG₂ and IgG₄. For instance, substitution of IgG₂ residues at positions 233-236 into human IgG₁ greatly reduced ADCC and CDC (Armour et al., 1999 and Shields et al., 2001).

Numerous mutations have been made in the CH2 domain of IgG and their effect on ADCC and CDC was tested in vitro (Shields et al., 2001, Idusogie et al., 2001 and 2000, Steurer et al., 1995). In particular, a mutation to alanine at E333 was reported to increase both ADCC and CDC (Idusogie et al., 2001 and 2000).

Increasing the serum half-life of a therapeutic antibody is another way to improve its efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG₁ (Hinton et al., 2004 and Vaccaro et al., 2005).

The following Table 1 summarizes some known mutations of the IgG Fc-region (taken from Invivogen's website).

| Engineered | IgG | | | | |
|---|---|---|---|---|---|
| Fc | Isotype | Mutations | Properties | Potential Benefits | Applications |
| hIgG1e1 | human | T250Q/M428L | Increased | Improved localization to | Vaccination; |
| | IgG1 | | plasma half- | target; increased | therapeutic |
| | | | life | efficacy; reduced dose | use |
| | | | | or frequency of | |
| | | | | administration | |
| hIgG1e2 | human | M252Y/S254T/T256E + | Increased | Improved localization to | Vaccination; |
| | IgG1 | H433K/N434F | plasma half- | target; increased | therapeutic us |
| | | | life | efficacy; reduced dose | |
| | | | | or frequency of | |
| | | | | administration | |
| hIgG1e3 | human | E233P/L234V/L235A/ | Reduced | Reduced adverse | Therapeutic |
| | IgG1 | ΔG236 + | ADCC and | events | use without |
| | | A327G/A330S/P331S | CDC | | cell depletion |
| hIgG1e4 | human | E333A | Increased | Increased efficacy | Therapeutic |
| | IgG1 | | ADCC and | | use with cell |
| | | | CDC | | depletion |
| hIgG2e1 | human | K322A | Reduced | Reduced adverse | Vaccination; |
| | IgG2 | | CDC | events | therapeutic |
| | | | | | use |

In certain known Fc-fusion proteins having therapeutic utility, Fc-regions have been fused to extracellular domains of certain receptors belonging to the tumor necrosis factor receptor (TNF-R) superfamily (Locksley et al., 2001, Bodmer et al., 2002, Bossen et al., 2006). A hallmark of the members of the TNFR family is the presence of cystein-rich pseudo-repeats in the extracellular domain, as described e.g. by Naismith and Sprang, 1998.

The two TNF receptors, p55 (TNFR1) and p75 TNFR (TNFR2) are examples of such members of the TNFR superfamily. Etanercept is an Fc-fusion protein containing the soluble part of the p75 TNFR (e.g. WO91/03553, WO 94/06476). Under the trade name Enbrel®, it is marketed for treatment of Endometriosis, Hepatitis C virus infection, HIV infection, Psoriatic arthritis, Psoriasis, Rheumatoid arthritis, Asthma, Ankylosing spondylitis, Cardiac failure, Graft versus host disease, Pulmonary fibrosis, Crohns disease. Lenercept is a fusion protein containing extracellular components of human p55 TNF receptor and the Fc portion of human IgG, and is intended for the potential treatment of severe sepsis and multiple sclerosis.

OX40 is also a member of the TNFR superfamily. OX40-IgG1 and OX40-hIG4mut fusion proteins have been prepared for treatment of inflammatory and autoimmune diseases such as Crohn's Disease.

An Fc-fusion protein of the BAFF-R, also called BR3, designated BR3-Fc, is a soluble decoy receptor from a series of inhibitors of BAFF (B-cell activating factor of the TNF family), is being developed for the potential treatment of autoimmune diseases such as rheumatoid arthritis (RA) and systemic lupus erythematosus (SLE).

BCMA is a further receptor belonging to the TNFR superfamily. A BCMA-Ig fusion protein has been described to inhibit autoimmune disease (Melchers, 2006).

Another receptor of the TNF-R superfamily is TACI, the transmembrane activator and CAML-interactor (von Bülow and Bram, 1997; U.S. Pat. No. 5,969,102, Gross et al., 2000), which has an extracellular domain containing two cysteine-rich pseudo-repeats. TACI binds two members of the tumor necrosis factor (TNF) ligand family. One ligand is designated BLyS, BAFF, neutrokine-α, TALL-1, zTNF4, or THANK (Moore et al., 1999). The other ligand has been designated as APRIL, TNRF death ligand-1 or ZTNF2 (Hahne et al., J. Exp. Med. 188: 1185 (1998).

Fusion proteins containing soluble forms of the TACI receptor fused to an IgG Fc region are known as well and were designated TACI-Fc (WO 00/40716, WO 02/094852). TACI-Fc inhibits the binding of BLyS and APRIL to B-cells (Xia et al., 2000). It is being developed for the treatment autoimmune diseases, including systemic lupus erythematosus (SLE), rheumatoid arthritis (RA) and hematological malignancies, as well as for treatment of multiple sclerosis (MS). In addition to this, TACI-Fc is being developed in multiple myeloma (MM) (Novak et al., 2004; Moreau et al., 2004) and non-Hodgkin's lymphoma (NHL), chronic lymphocytic leukemia (CLL) and Waldenstrom's macroglobulemia (WM).

Given the therapeutic utility of Fc-fusion proteins, in particular those containing extracellular portions of the TNFR superfamily, there is a need for significant amounts of highly purified protein that is adequate for human administration.

WO 02/094852 describes a method for partially purifying TACI-Fc, which comprises protein A chromatography followed by S-200 size exclusion chromatography.

WO 03/059935 discloses a purification process for a p75 TNFR:Fc-fusion protein using a combination of hydroxyapatite chromatography and affinity chromatography on Protein A. However, in the process described in WO 03/059935, the Fc-fusion protein does not bind to hydroxyapatite and is thus contained in the flow-through of the hydroxyapatite column. In addition to this, use of ion exchange chromatography is not mentioned for purification of the p75 TNFR:Fc-fusion protein.

WO 2005/044856 discloses a method for removing high molecular weight aggregates from antibody preparations by hydroxyapatite chromatography. A purification method using Protein A, anion exchange chromatography and hydroxyapatite chromatography is disclosed as well. However, firstly this method has been described exclusively for antibodies and secondly, there is no disclosure of the use of a cation exchange chromatography step between the Protein A affinity and the anion exchange step.

WO 94/06476 proposes hypothetical purification protocols for recombinant soluble TNF receptors based on TNF or lectin affinity chromatography, anion or cation exchange chromatography and reverse-phase high performance liquid chromatography (RP-HPLC). Hydroxyapatite chromatography is not mentioned in this document as a suitable purification step for soluble TNF receptors.

US 2002/0115175 describes purification of metalloproteases such as TNF alpha convertase enzyme. TACE has a theoretical isolelectric point of approximately 5.4, as calculated e.g. using the “EMBL WWW Gateway to Isoelectric Point Service”, available on the internet. TACE is a protease that cleaves 8 amino acids off at the N-terminus of membrane bound (pro-) TNF alpha. The cytokine TNF alpha is thus released from the cell membrane and thereby activated. The process for purification of TACE disclosed in US 2002/0115175 contains a step on wheat germ agglutinin agarose. Fc fusion proteins of TACE are described in this document as well, but have not been purified.

EP 1 561 756 discloses that protein A or G based chromatography alone may not be sufficient for the separation of DNA contaminants from proteins and that in order to purify a protein, further steps such as anion or cation exchange chromatography, hydroxyapatite chromatography or combinations thereof may be used. No specific order has been proposed for these chromatographic steps. Additionally, the proteins EP 1 561 756 refers to are hematopoietic factors, cytokines and antibodies. Fc-fusion proteins are not mentioned in EP 1 561 756.

EP 1 614 693 describes a method for purification of antibodies based on protein A affinity chromatography, anion exchange chromatography and cation exchange chromatography. In this document, it is specified that the antibodies are purified via anion exchange and cation exchange chromatography in that order, or, alternatively, via cation exchange chromatography followed by hydrophobic chromatography. The hydrophobic chromatography may be replaced by any other type of chromatography including hydroxyapatite chromatography. Fc-fusion proteins are not mentioned in EP 1 614 693.

Feng et al., 2005, disclose methods for the purification of antibodies based on an initial capture step on Protein A followed by polishing steps that may be hydrophobic interaction chromatography, anion exchange chromatography, cation exchange chromatography or, hydroxyapatite chromatography. However, Feng et al. only describe methods for antibody purification and not for Fc-fusion proteins. In addition to this, apart from the initial Protein A affinity step, no specific order is suggested in order to systematically remove all unwanted impurities such as host cell proteins (HCPs), aggregates, DNA, viral contaminants and leached Protein A.

Therefore, there is still an unmet need for efficient purification methods for Fc-fusion proteins resulting in such purity as to be suitable for human administration.

### SUMMARY OF THE INVENTION

The present invention is based on the development of a purification process for an Fc-fusion protein.

Therefore, in a first aspect, the invention relates to a process for the purification of an Fc-fusion protein, comprising the following steps:
a. Subjecting a fluid comprising said Fc-fusion protein to Protein A or Protein G affinity chromatography;
b. Subjecting the eluate of step (a) to Cation exchange chromatography;
c. Subjecting the eluate of step (b) to Anion exchange chromatography; and
d. Subjecting the flow-through of step (c) to Hydroxyapatite chromatography and collecting the eluate to obtain purified Fc-fusion protein.

This process is used for purifying Fc-fusion proteins having an isoelectric point (pI) in the range of between 7.0 and 9.5.

The process is preferably used for purifying therapeutic Fc-fusion proteins, i.e. Fc-fusion proteins intended for human administration. More preferably, it is used for an Fc-fusion protein comprising an extracellular portion, in particular a ligand binding and optionally inhibiting extracellular portion, of a member of the tumor necrosis factor receptor (TNFR) superfamily.

It has been surprisingly shown that step (b) was suitable for removal of so-called free Fc, i.e. immunoglobulin heavy chain domains which are not fused to a complete therapeutic moiety such as e.g. a ligand binding extracellular portion of a member of the TNFR family.

In a second aspect, the invention relates to a purified Fc-fusion protein, preferably a therapeutic Fc-fusion protein, more preferably an Fc-fusion protein comprising an extracellular portion, in particular a ligand binding extracellular portion, of a member of the tumor necrosis factor receptor (TNFR) superfamily, comprising less than 1% or 0.5% or 0.2% or 0.1% of free Fc protein.

It has further been shown that the combination of steps (a), (c) and (d) significantly eliminated Fc-fusion protein aggregates, which are therapeutically inactive and not desirable for human administration.

Therefore, in a third aspect, the invention relates to a purified Fc-fusion protein composition, preferably a therapeutic Fc-fusion protein, more preferably an Fc-fusion protein comprising an extracellular portion, in particular a ligand binding extracellular portion, of a member of the tumor necrosis factor receptor (TNFR) superfamily, comprising less than 1% or less than 0.5% of Fc-fusion protein aggregates and/or less than 0.5% or less than 0.2% or less than 0.1% of free Fc protein.

A further aspect of the present invention relates to the use of cation exchange chromatography for the removal of free Fc in an Fc-fusion protein preparation, preferably a therapeutic Fc-fusion protein preparation, more preferably of a Fc-fusion protein comprising an extracellular portion of a member of the tumor necrosis factor receptor family, or a ligand binding and optionally inhibiting fragment thereof.

Yet a further aspect of the present invention relates to the use of hydroxyapatite chromatography for the removal of aggregates in an Fc-fusion protein preparation, preferably therapeutic Fc-fusion protein preparations, more preferably Fc-fusion proteins comprising an extracellular portion of a member of the tumor necrosis factor receptor (TNFR) superfamily, or a ligand binding fragment thereof.

### BRIEF DESCRIPTION OF THE DRAWINGS

FIG. 1 shows a non-reduced silver stained SDS-PAGE of different fractions stemming from the cation exchange chromatography described in Example 2. Lane 1: Molecular weight markers, Lane 2: purified TACI-Fc, Lane 3: load, Lane 4: wash 2, Lane 5: eluate 2, Lane 6: wash 3, Lane 7: eluate 3, Lane 8: wash 1, Lane 9: eluate 1, Lane 10: purified free Fc;

FIG. 2 shows the chromatographic profile of the cation exchange chromatography described in Example 2.

### BRIEF DESCRIPTION OF THE SEQUENCE LISTING

SEQ ID NO: 1 is a cysteine fingerprint sequence (cysteine rich pseudo repeat) common to members of the TNFR superfamily;
SEQ ID NO: 2 is the full length sequence of the human TACI receptor (e.g. described in WO 98/39361);
SEQ ID NO: 3 is an example of a human Fc sequence of the invention (e.g. described in WO 02/094852);
SEQ ID NO: 4 is a preferred Fc-fusion protein of the invention, comprising sequences derived from the extracellular portion of TACI and a human IgG, Fc portion (e.g. described in WO 02/094852);
SEQ ID NO: 5 is a polynucleotide coding for a polypeptide of SEQ ID NO: 2 (e.g. described in WO 02/094852);
SEQ ID NO: 6 is a polynucleotide coding for a polypeptide of SEQ ID NO: 3 (e.g. described in WO 02/094852);
SEQ ID NO: 7 is a polynucleotide coding for a polypeptide of SEQ ID NO: 4 (e.g. described in WO 02/094852).

### DETAILED DESCRIPTION OF THE INVENTION

The present invention is based on the development of a purification method for an exemplary therapeutic Fc-fusion protein, named TACI-Fc, resulting in a highly purified TACI-Fc preparation that is suitable for human administration.

The invention therefore relates to a method for purifying an Fc-fusion protein comprising the following steps:
a. Subjecting a fluid comprising said Fc-fusion protein to Protein A or Protein G affinity chromatography;
b. Subjecting the eluate of step (a) to Cation exchange chromatography;
c. Subjecting the eluate of step (b) to Anion exchange chromatography;
d. Subjecting the flow-through of step (c) to Hydroxyapatite chromatography and collecting the eluate to obtain purified Fc-fusion protein.

In an embodiment of the invention, the purification method does not contain a step on lectin affinity chromatography, and in particular it does not comprise a step on wheat germ agglutinin agarose.

The method of the invention is used for purifying an Fc-fusion protein having a pI ranging from 6.9 to 9.5. The “isoelectric point” or “pI” of a protein is the pH at which the protein has a net overall charge equal to zero, i.e. the pH at which the protein has an equal number of positive and negative charges. Determination of the pI for any given protein can be done according to well-established techniques, such as e.g. by isoelectric focusing.

The pI of the Fc-fusion protein to be purified in accordance with the present invention can thus be e.g. any of 6.9, 6.95, 7.0, 7.05, 7.1, 7.15, 7.2, 7.25, 7.3, 7.35, 7.4, 7.45, 7.5, 7.55, 7.6, 7.65, 7.7, 7.75, 7.8, 7.85, 7.9, 7.95, 8.0, 8.05, 8.1, 8.15, 8.2, 8.25, 8.3, 8.35, 8.4, 8.45, 8.5, 8.55, 8.6, 8.65, 8.7, 8.75, 8.8, 8.85, 8.9, 8.95, 9.0, 9.05, 9.1, 9.15, 9.2, 9.25, 9.3, 9.35, 9.4, 9.45, 9.5.

Preferably, the pI of the Fc-fusion protein to be purified in accordance with the present invention is 8 to 9 or 8.0 to 9.0, more preferably 8.3 to 8.6.

The method of the invention is preferably for purifying a therapeutic Fc-fusion protein, i.e. an Fc-fusion protein intended for treatment or prevention of disease of an animal or preferably for human treatment. More preferably, the method of the invention is for purifying an Fc-fusion protein comprising an extracellular portion of a member of the tumor necrosis factor receptor (TNFR) superfamily. The extracellular portion is preferably a ligand binding fragment of an extracellular part or domain of the respective receptor. A preferred Fc-fusion protein that can be purified in accordance with the invention binds ligand and inhibits or blocks ligand function, e.g. receptor activation.

The term “Fc-fusion protein”, as used herein, is meant to encompass proteins, in particular therapeutic proteins, comprising an immunoglobulin-derived moiety, which will be called herein the “Fc-moiety”, and a moiety derived from a second, non-immunoglobulin protein, which will be called herein the “therapeutic moiety”, irrespective of whether or not treatment of disease is intended.

The term “free Fc”, as used herein, is meant to encompass any part of the Fc-fusion protein to be purified in accordance with the present invention, which is derived from the immunoglobulin part of the Fc-fusion protein and does not contain a significant portion of the therapeutic moiety of the Fc-fusion protein. Therefore, free Fc may contain dimers of the IgG hinge, CH2 and CH3 domains, which are not linked or bound to significant portions of a therapeutic moiety, corresponding e.g. to the Fc part that is generated by papain cleavage. Monomers derived from the Fc-moiety may also be contained in the free Fc fraction. It is understood that free Fc may still contain a number of amino acid residues from the therapeutic moiety, such as e.g. one to ten (e.g. 2, 3, 4, 5, 6, 7, 8 or 9) amino acids belonging to the therapeutic moiety, fused to the Fc-moiety.

The Fc-moiety may be derived from a human or animal immunoglobulin (Ig) that is preferably an IgG. The IgG may be an IgG₁, IgG₂, IgG₃ or IgG₄. It is also preferred that the Fc-moiety is derived from the heavy chain of an immunoglobulin, preferably an IgG. More preferably, the Fc-moiety comprises a portion, such as e.g. a domain, of an immunoglobulin heavy chain constant region. Such Ig constant region preferably comprises at least one Ig constant domain selected from any of the hinge, CH2, CH3 domain, or any combination thereof. It is preferred that the Fc-moiety comprises at least a CH2 and CH3 domain. It is further preferred that the Fc-moiety comprises the IgG hinge region, the CH2 and the CH3 domain.

The Fc-fusion protein of the invention may be a monomer or dimer. The Fc-fusion protein may also be a “pseudo-dimer”, containing a dimeric Fc-moiety (e.g. a dimer of two disulfide-bridged hinge-CH2-CH3 constructs), of which only one is fused to a therapeutic moiety.

The Fc-fusion protein may be a heterodimer, containing two different therapeutic moieties, or a homodimer, containing two copies of a single therapeutic moiety.

In accordance with the present invention, the Fc-moiety may also be modified in order to modulate effector functions. For instance, the following Fc mutations, according to EU index positions (Kabat et al., 1991), can be introduced if the Fc-moiety is derived from IgG₁:

T250Q/M428L

M252Y/S254T/T256E+H433K/N434F

E233P/L234V/L235A/AΔ236+A327G/A330S/P331S

E333A; K322A.

Further Fc mutations may e.g. be the substitutions at EU index positions selected from 330, 331 234, or 235, or combinations thereof. An amino acid substitution at EU index position 297 located in the CH2 domain may also be introduced into the Fc-moiety in the context of the present invention, eliminating a potential site of N-linked carbohydrate attachment. The cysteine residue at EU index position 220 may also be replaced with a serine residue, eliminating the cysteine residue that normally forms disulfide bonds with the immunoglobulin light chain constant region.

In accordance with the present invention, it is preferred that the Fc-moiety comprises or consists of SEQ ID NO: 3 or is encoded by a polynucleotide comprising SEQ ID NO: 6.

The therapeutic moiety of the invention may e.g. be or be derived from EPO, TPO, Growth Hormone, Interferon-alpha, Interferon-beta, Interferon-gamma, PDGF-beta, VEGF, IL-1alpha, IL-1beta, IL-2, IL-4, IL-5, IL-8, IL-10, IL-12, IL-18, IL-18 binding protein, TGF-beta, TNF-alpha, or TNF-beta.

The therapeutic moiety of the invention may also be derived from a receptor, e.g a transmembrane receptor, preferably be or be derived from the extracellular domain of a receptor, and in particular a ligand binding and optionally inhibiting fragment of the extracellular part or domain of a given receptor. Examples for therapeutically interesting receptors are CD2, CD3, CD4, CD8, CD11a, CD14, CD18, CD20, CD22, CD23, CD25, CD33, CD40, CD44, CD52, CD80, CD86, CD147, CD164, IL-2 receptor, IL-4 receptor, IL-6 receptor, IL-12 receptor, IL-18 receptor subunits (IL-18R-alpha, IL-18R-beta), EGF receptor, VEGF receptor, integrin alpha 4 10 beta 7, the integrin VLA4, B2 integrins, TRAIL receptors 1, 2, 3, and 4, RANK, RANK ligand, epithelial cell adhesion molecule (EpCAM), intercellular adhesion molecule-3 (ICAM-3), CTLA4 (which is a cytotoxic T lymphocyte-associated antigen), Fc-gamma-I receptor, HLA-DR 10 beta, HLA-DR antigen, L-selectin.

It is highly preferred that the therapeutic moiety of the invention be derived from a receptor belonging to the TNFR superfamily. The therapeutic moiety may e.g. be or be derived from the extracellular domain of TNFR1 (p55), TNFR2 (p75), OX40, Osteoprotegerin, CD27, CD30, CD40, RANK, DR3, Fas ligand, TRAIL-R1, TRAIL-R2, TRAIL-R3, TAIL-R4, NGFR, AITR, BAFFR, BCMA, TACI.

In accordance with the present invention, the therapeutic moiety derived from a member of the TNFR superfamily preferably comprises or consists of all or part of the extracellular domain of the member of the TNFR, and more preferably comprises a ligand binding and optionally inhibiting fragment of such a member of the TNFR.

The following Table 5 lists members of the TNFR superfamily from which a therapeutic moiety in accordance with the present invention may be derived, and their respective ligands. A “ligand binding fragment” of a member of the TNFR family can easily be determined by the person skilled in the art, e.g. in a simple in vitro assay measuring binding between protein fragment of a given receptor and the respective ligand. Such an assay can e.g. be a simple in vitro RIA- or ELISA-type sandwich assay wherein one of the proteins, e.g. the receptor fragment, is immobilized to a carrier (e.g. an ELISA plate) and is incubated, following appropriate blocking of the protein binding sites on the carrier, with the second protein, e.g. the ligand. After incubation, ligand binding is detected e.g. by way of radioactive labeling of the ligand and determination of the bound radioactivity, after appropriate washing, in a scintillation counter. Binding of the ligand can also be determined with a labeled antibody, or a first ligand-specific antibody and a second, labeled antibody directed against the constant part of first antibody. Ligand binding can thus be easily determined, depending of the label used, e.g. in a color reaction. Blocking or inhibition of ligand binding function can be tested in suitable cell-based assays.

Preferably, the method of the present invention is for purifying an Fc-fusion protein comprising a therapeutic moiety derived from a member of the TNFR superfamily selected from those listed in Table 5.

TABLE 5
The TNFR superfamily
(according to Locksley et al., 2001 and Bossen et al., 2006)

| Member of TNFR superfamily | Ligand |
|---|---|
| NGFR | NGF |
| EDAR | EDA-A1 |
| XEDAR | EDA-A2 |
| CD40 | CD40L |
| Fas | FasL |
| Ox40 | OX40L |
| AITR | AITRL |
| GITR | GITRL |
| CD30 | CD30L |
| CD40 | CD40L |
| HveA | LIGHT, LT-alpha |
| 4-1BB | 4-1BBL |
| TNFR2 | TNF-alpha, LT-alpha, LT-alpha-beta |
| LT-betaR | LIGHT, LT-alpha, LT-alpha-beta |
| DR3 | TL1A |
| CD27 | CD27L |
| TNFR1 | TNF-alpha, LT-alpha, LT-alpha-beta |
| LTBR | LT-beta |
| RANK | RANKL |
| TACI | BlyS, APRIL |
| BCMA | BlyS, APRIL |
| BAFF-R | BAFF (=BlyS) |
| TRAILR1 | TRAIL |
| TRAILR2 | TRAIL |
| TRAILR3 | TRAIL |
| TRAILR4 | TRAIL |
| Fn14 | TWEAK |
| OPG | RANKL, TRAIL |
| DR4 | TRAIL |
| DR5 | TRAIL |
| DcR1 | TRAIL |
| DcR2 | TRAIL |
| DcR3 | FasL, LIGHT, TL1A |

In a preferred embodiment, the Fc-fusion protein comprises a therapeutic moiety selected from an extracellular domain of TNFR1, TNFR2, or a TNF binding and optionally inhibiting fragment thereof.

In a further preferred embodiment, the Fc-fusion protein comprises a therapeutic moiety selected from an extracellular domain of BAFF-R, BCMA, or TACI, or a fragment thereof binding at least one of Blys or APRIL.

An assay for testing the capability of binding to Blys or APRIL is described e.g. in Hymowitz et al., 2006.

TACI is preferably human TACI. SEQ ID NO: 2 corresponds to the amino acid sequence of human full-length TACI receptor (also SwissProt entry 014836). More preferably, the therapeutic moiety comprises a soluble portion of TACI, preferably derived from the extracellular domain of TACI. Preferably, the TACI-derived therapeutic moiety comprises at least amino acids 33 to 67 of SEQ ID NO: 2 and/or amino acids 70 to 104 of SEQ ID NO: 2. In a preferred embodiment, the TACI extracellular domain included in the therapeutic moiety according to the invention comprises or consist of amino acids 1 to 166 of SEQ ID NO: 2 or amino acids 30 to 166 of SEQ ID NO: 2, or amino acids 30 to 119 of SEQ ID NO: 2, or amino acids 30 to 110 of SEQ ID NO: 2. All of those therapeutic moieties are preferred for the preparation of the Fc-fusion protein to be purified by the method of the invention and are combined with the Fc-moieties described in detail above, and in particular with an Fc-moiety comprising or consisting of SEQ ID NO: 3. A highly preferred Fc-fusion protein to be purified in accordance with the present invention comprises or consists of SEQ ID NO: 4 or encoded by the polynucleotide of SEQ ID NO: 7.

Hence, it is highly preferred that the Fc-fusion protein comprises a polypeptide selected from
a. amino acids 34 to 66 of SEQ ID NO: 2;
b. amino acids 71 to 104 of SEQ ID NO: 2;
c. amino acids 34 to 104 of SEQ ID NO: 2;
d. amino acids 30 to 110 of SEQ ID NO: 2;
e. SEQ ID NO: 3;
f. SEQ ID NO: 4;
g. a polypeptide encoded by a polynucleotide hybridizing to the complement of SEQ ID NO: 5 or 6 or 7 under highly stringent conditions; and
h. a mutein of any of (c), (d), (e), or (f) having at least 80% or 85% or 90% or 95% sequence identity to the polypeptide of (c), (d), (e) or (f);

wherein the polypeptide binds to at least one of Blys or APRIL.

In a further preferred embodiment, the Fc-fusion protein comprises a heavy chain constant region of an immunoglobulin, more preferably a human constant region. n an embodiment of the invention, the immunoglobulin is an IgG₁. It is also preferred that the constant region comprises a hinge, CH2 and a CH3 domain.

In a further embodiment, the therapeutic moiety comprises the cysteine rich pseudo-repeat of SEQ ID NO: 1.

In accordance with the present invention, a fluid comprising an Fc-fusion protein is first subjected to Protein A or Protein G affinity chromatography. The fluid may preferably be cell culture material, e.g. solubilized cells, more preferably cell culture supernatant. The term “cell culture supernatant”, as used herein, refers to a medium in which cells are cultured and into which proteins are secreted provided they contain appropriate cellular signals, so-called signal peptides. It is preferred that the Fc-fusion protein expressing cells are cultured under serum-free culture conditions. Thus, preferably, the cell culture supernatant is devoid of animal-serum derived components. Most preferably, the cell culture medium is a chemically defined medium.

The Protein A used for the affinity chromatography may e.g. be recombinant. It may also be modified in order to improve its properties (such as e.g. in the resin called MabSelect SuRe, commercially available from GE Healthcare). In a preferred embodiment, step (a) is carried out on a resin comprising cross-linked agarose modified with recombinant Protein A. A column commercially available under the name Mabselect Xtra (from GE Healthcare) is an example of an affinity resin that is particularly suitable for step (a) of the present method.

The Protein A or G affinity chromatography is preferably used as a capture step, and thus serves for purification of the Fc-fusion protein, in particular elimination of host cell proteins and Fc-fusion protein aggregates, and for concentration of the Fc-fusion protein preparation.

The term “aggregates”, as used herein, is meant to refer to protein aggregates. It encompasses multimers (such as dimers, tetramers or higher order aggregates) of the Fc-fusion protein to be purified and may result e.g. in high molecular weight aggregates.

The affinity chromatography has the further advantage of reducing aggregate levels by 2 to 4 fold.

In using the Protein A or G affinity chromatography, host cell protein levels may be reduced by 100 to 300 fold.

In a preferred embodiment of the invention, the elution in step (a) is carried out at a pH ranging from 2.8 to 4.5, preferably from 3.0 to 4.2, more preferably at 3.5, 3.55, 3.6, 3.65, 3.7, 3.75, 3.8, 3.85, 3.9, 3.95, 4.0, 4.05, 4.1, or 4.15. The elution in step (a) may also be carried out with a pH gradient, preferably a gradient from pH 4.5 to 2.8.

In a further preferred embodiment, the elution in step (a) is carried out in a buffer selected from sodium acetate or sodium citrate. Suitable buffer concentrations are e.g. selected from 50 mM or 100 mM or 150 mM or 200 mM or 250 mM.

In accordance with the present invention, the eluate from the Protein A or Protein G chromatography is subjected to cation exchange chromatography. The cation exchange chromatography may be carried out on any suitable cation exchange resin, such as e.g. weak or strong cation exchangers as explained above in the Background of the Invention.

Preferably, step (b) is carried out on a strong cation exchange resin. More preferably, the cation exchange material comprises a cross-linked methacrylate modified with SO₃⁻ groups. A column commercially available under the name Fractogel EMD SO₃⁻ (from Merck) is an example of a cation exchange resin that is particularly suitable for step (b) of the present method.

Preferably, the Protein A eluate is loaded directly on the cation exchange column. It is preferred that loading is carried out at a pH of at least one unit below the pI of the Fc-fusion protein to be purified.

It is further preferred that after loading, the column is washed with a buffer having a conductivity of 6 to 10 mS/cm, e.g. at 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, or 9.9 mS/cm. More preferably, the conductivity ranges from 7.6 to 9.2, i.e. 8.4±0.8 mS/cm. The washing step is preferably carried out at a pH ranging from 5.5 to 7.5, preferably from 6.0 to 7.0.

In a further preferred embodiment, the cation exchange column is eluted at a pH ranging from 7.0 to 8.5, preferably 7.25 or 7.3 or 7.35 or 7.4 or 7.45 or 7.5 or 7.55 or 7.6 or 7.65 or 7.7 or 7.7 or 7.75 or 7.8 or 7.85 or 7.9 or 7.95 or 8.0 or 8.05 or 8.1 or 8.15 or 8.2 or 8.25 or 8.3 or 8.35 or 8.4 or 8.45 or 8.5.

Elution may preferably be carried out at a conductivity ranging from 15 to 22 mS/cm. For instance, the conductivity may be selected from 16, 17, 18, 19, 20, 21, or 22 mS/cm. A preferred buffer for elution is a phosphate buffer.

In a highly preferred embodiment, step (b) comprises the following further steps:
b.1. Washing the cation exchange resin with a buffer having a pH ranging from 6.0 to 7.0 and a conductivity ranging from 6 to 10 mS/cm; and
b.2. Eluting the column at a pH ranging from 7.0 to 8.5 and a conductivity ranging from 15 to 22 mS/cm.

A preferred buffer for step (b.1) is 75 to 125 mM sodium phosphate.

It has been surprisingly found in the frame of the present invention that step (b) efficiently eliminates free Fc. Therefore, in accordance with the present invention, cation exchange chromatography can preferably be used for elimination or reduction of free Fc in the range of 5 to 15 fold.

Advantageously, step (b) of the method of the present invention also reduces the concentration of host cell proteins from the Fc-fusion protein preparation, e.g. in the range of 1 to 2 fold, thus contributing significantly to the host cell protein (HCP) clearance.

In accordance with the present invention, the eluate from the cation exchange step is then subjected to an anion exchange chromatography. The anion exchange chromatography may be carried out on any suitable anion exchange resin, such as e.g. weak or strong anion exchangers as explained above in the Background of the Invention. Preferably, step (c) is carried out on a strong anion exchange resin. More preferably the anion exchange resin comprises polystyrene/divinyl benzene modified with N⁺(CH₃)₃. A column commercially available under the name Source 30Q (from GE Healthcare) is an example of an anion exchange resin that is particularly suitable for step (c) of the present method.

Preferably, the eluate of step (b) is diluted or dialysed into an appropriate loading buffer before loading it on the anion exchange column. The anion exchange column is also preferably equilibrated with the loading buffer.

A preferred pH for the loading buffer is one unit below the pI. Suitable pH values range from 6.0 to 8.5, preferably from 7.0 to 8.0, e.g. 7.0, 7.05, 7.1, 7.15, 7.2, 7.25, 7.3, 7.35, 7.4, 7.45, 7.5, 7.55, 7.6, 7.65, 7.7, 7.75, 7.8, 7.85, 7.9, 7.95, or 8.0. A preferred conductivity for the loading buffer is in the range of 3.0 to 4.6 mS/cm.

An appropriate equilibration/loading buffer may e.g. be sodium phosphate at a concentration ranging from 5 to 35, preferably from 20 to 30 mM. The buffer concentration may e.g. be at 10, 15, 20, 25, 30 mM. In the frame of the present invention, the flow-through (also called break-through) of the anion exchange chromatography, comprising the Fc-fusion protein of interest, is being collected.

Step (c) of the method of the invention further reduces aggregates 3 to 5 fold and host cell proteins 30 to 70 fold.

In accordance with the present invention, the flow-through of the anion exchange chromatography of step (c) is then used for further purification by hydroxyapatite chromatography. Any hydroxyapatite resin may be used to carry out step (d) of the method according to the invention. In a preferred embodiment, step (d) is carried out on a ceramic hydroxyapatite resin, such as a type I or type II hydroxyapatite resin. The hydroxyapatite resin may have particles of any size such as 20, 40 or 80 μm. In a highly preferred embodiment, the ceramic hydroxyapatite resin comprises particles having a size of 40 μm. A hydroxyapatite resin that is particularly suitable for step (d) of the present method is a column commercially available under the name CHT Ceramic Hydroxyapatite Type I, 40 μm.

In a preferred embodiment, the flow-through from step (c) is directly loaded on the hydroxyapatite resin, i.e. without previous dilution or dialysis into an appropriate loading buffer. Loading is preferably carried out at a pH of 6.5 to 7.5, such as 6.6, 6.7, 6.8, 6.9, 7.1, 7.2, 7.3, or 7.4, and preferably 7.0.

In a further preferred embodiment, the elution in step (d) is carried out in the presence of sodium phosphate ranging from 2 to 10 mM, preferably ranging from 1.75 to 5.25 mM, such as e.g. at 2, 2.25, 2.5, 2.75, 3, 3.25, 3.5, 3.75, 4, 4.25, 4.5, 4.75, 5.

In yet a further preferred embodiment, the elution in step (d) is carried out at a pH ranging from 6.0 to 7.0, e.g. at 6.1, 6.2, 6.3, 6.4, 6.5, 6.6, 6.7, 6.8, 6.9.

In another preferred embodiment, elution in step (d) is carried out in the presence of potassium chloride ranging from 0.4 to 1 M, preferably between 0.45, 0.5, 0.55, 0.6, 0.65, 0.7, 0.75, 0.8, 0.85, 0.9, 0.95 M, most preferably at 0.6 M.

In accordance with the present invention, the eluate of step (d) is collected, containing the finally purified Fc-fusion protein preparation.

Suitable matrix materials, i.e. carrier materials for the chromatographic resins used in steps (a) to (c), that may be used in connection with the present invention may e.g. be agarose (sepharose, superose) dextran (sephadex), polypropylene, methacrylate cellulose, polystyrene/divinyl benzene, or the like. The resin materials may be present in different cross-linked forms, depending on the specific use.

The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

In a preferred embodiment of the present purification process, one or more ultrafiltration steps are performed. Ultrafiltration is useful for removal of small organic molecules and salts in the eluates resulting from previous chromatrographic steps, to equilibrate the Fc-fusion protein in the bulk buffer, or to concentrate the Fc-fusion protein to the desired concentration. Such ultrafiltration may e.g. be performed on ultrafiltration membranes, with pore sizes allowing the removal of components having molecular weights below 5, 10, 15, 20, 25, 30 or more kDa.

Preferably, ultrafiltration is carried out between steps (b) and (c), and/or after step (d). More preferably, two ultrafiltration steps are carried out, one between steps (b) and (c) and one after step (d).

If the protein purified according to the process of the invention is intended for administration to humans, it is advantageous to include one or more steps of virus removal in the process. Preferably, a virus removal filtration step is carried out after step (d). More preferably, the virus removal filtration step is a nanofiltration step where the filter has a nominal pore size of 20 nm. The method of the present invention, and in particular steps (a), (c), (d) in combination with nanofiltration efficiently eliminates virus load to a combined LRV (log reduction value) of up to about 15 to 25.

In order to facilitate storage or transport, for instance, the material may be frozen and thawed before and/or after any purification step of the invention.

In accordance with the present invention, the recombinant Fc-fusion protein may be produced in eukaryotic expression systems, such as yeast, insect, or mammalian cells, resulting in glycosylated Fc-fusion proteins.

In accordance with the present invention, it is most preferred to express the Fc-fusion protein in mammalian cells such as animal cell lines, or in human cell lines. Chinese hamster ovary cells (CHO) or the murine myeloma cell line NSO are examples of cell lines that are particularly suitable for expression of the Fc-fusion protein to be purified. The Fc-fusion protein can also preferably be produced in human cell lines, such as e.g. the human fibrosarcoma HT1080 cell line, the human retinoblastoma cell line PERC6, or the human embryonic kidney cell line 293, or a permanent amniocyte cell line as described e.g. in EP 1 230 354.

If the Fc-fusion protein to be purified is expressed by mammalian cells secreting it, the starting material of the purification process of the invention is cell culture supernatant, also called harvest or crude harvest. If the cells are cultured in a medium containing animal serum, the cell culture supernatant also contains serum proteins as impurities.

Preferably, the Fc-fusion protein expressing and secreting cells are cultured under serum-free conditions. The Fc-fusion protein may also be produced in a chemically defined medium. In this case, the starting material of the purification process of the invention is serum-free cell culture supernatant that mainly contains host cell proteins as impurities. If growth factors are added to the cell culture medium, such as insulin, for example, these proteins will be eliminated during the purification process as well.

In order to create soluble, secreted Fc-fusion proteins, that are released into the cell culture supernatant, either the natural signal peptide of the therapeutic moiety of the Fc-fusion protein is used, or preferably a heterologous signal peptide, i.e. a signal peptide derived from another secreted protein being efficient in the particular expression system used, such as e.g. the bovine or human Growth Hormone signal peptide, or the immunoglobulin signal peptide.

As mentioned above, a preferred Fc-fusion protein to be purified in accordance with the present invention is a fusion protein having a therapeutic moiety derived from human TACI (SEQ ID NO: 2), and in particular a fragment derived from its extracellular domain (amino acids 1 to 165 of SEQ ID NO: 2). A preferred fragment comprises amino acids 30 to 110 of SEQ ID NO: 2. In the following, therapeutic moieties derived from the extracellular domain of TACI will be called “soluble TACI” or “sTACI”. A preferred Fc-moiety comprises SEQ ID NO: 3, resulting in an Fc-fusion protein according to SEQ ID NO: 4, in the following called “TACI-Fc”.

The term TACI-Fc, as used herein, also encompasses muteins of TACI-Fc.

The term “muteins”, as used herein, refers to analogs of sTACI or TACI-Fc, in which one or more of the amino acid residues of sTACI or TACI-Fc are replaced by different amino acid residues, or are deleted, or one or more amino acid residues are added to the original sequence of sTACI or TACI-Fc without changing considerably the activity of the resulting products as compared with the original sTACI or TACI-Fc. These muteins are prepared by known synthesis and/or by site-directed mutagenesis techniques, or any other known technique suitable therefor.

Muteins in accordance with the present invention include proteins encoded by a nucleic acid, such as DNA or RNA, which hybridizes to the complement of a DNA or RNA, which encodes a sTACI or TACI-Fc according to any of SEQ ID NOs: 2 or 4 under stringent conditions. An example for a DNA sequence encoding a TACI-Fc is SEQ ID NO: 7.

The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992). Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

In another embodiment, any such mutein has at least 50%, at least 60%, at least 70%, at least 75%, at least 80%, at least 85%, at least 90%, or at least 95% identity or homology thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990).

Any such mutein preferably has a sequence of amino acids sufficiently duplicative of that of sTACI or TACI-Fc, such as to have substantially similar ligand binding activity as a protein of SEQ ID NO: 2 or 4. For instance, one activity of TACI is its capability of binding to Blys or APRIL (Hymowitz et al., 2006). As long as the mutein has substantial APRIL or Blys binding activity, it can be considered to have substantially similar activity to TACI. Thus, it can be easily determined by the person skilled in the art whether any given mutein has substantially the same activity as a protein of SEQ ID NO: 2 or 4 by means of routine experimentation.

Preferred changes for muteins in accordance with the present invention are what are known as “conservative” substitutions. Conservative amino acid substitutions of sTACI or TACI-Fc, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, under twenty, or preferably under ten, and do not remove or displace amino acids which are critical to a functional conformation, e.g., cysteine residues. Proteins and muteins produced by such deletions and/or insertions come within the purview of the present invention.

Preferably, the conservative amino acid groups are those defined in Table 2. More preferably, the synonymous amino acid groups are those defined in Table 3; and most preferably the synonymous amino acid groups are those defined in Table 4.

TABLE 2
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE 3
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE 4
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

A functional derivative may be prepared from an Fc-fusion protein purified in accordance with the present invention. “Functional derivatives” as used herein cover derivatives of the Fc-fusion protein to be purified in accordance with the present invention, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of the unmodified Fc-fusion protein as defined above, and do not confer toxic properties on compositions containing it.

Functional derivatives of an Fc-fusion protein can e.g. be conjugated to polymers in order to improve the properties of the protein, such as the stability, half-life, bioavailability, tolerance by the human body, or immunogenicity. To achieve this goal, TACI-Fc may be linked e.g. to polyethylene glycol (PEG). PEGylation may be carried out by known methods, described in WO 92/13095, for example.

Functional derivatives may also, for example, include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

In a third aspect, the invention relates to a protein purified by the process of purification according to the invention. In the following, such protein is also called “purified Fc-fusion protein”.

Such purified Fc-fusion protein is preferably highly purified Fc-fusion protein. Highly purified Fc-fusion protein is determined e.g. by the presence of a single band in a silver-stained, non-reduced SDS-PAGE-gel after loading of protein in the amount of 2 mcg per lane. Purified Fc-fusion protein may also be defined as eluting as a single peak in HPLC.

The Fc-fusion protein preparation obtained from the purification process of the invention may contain less than 20% of impurities, preferably less than 10%, 5%, 3%, 2% or 1% of impurities, or it may be purified to homogeneity, i.e. being free from any detectable proteinaceous contaminants as determined e.g. by silver stained SDS-PAGE or HPLC, as explained above.

Purified Fc-fusion proteins may be intended for therapeutic use, in particular for administration to human patients. If purified Fc-fusion protein is administered to patients, it is preferably administered systemically, and preferably subcutaneously or intramuscularly, or topically, i.e. locally. Rectal or intrathecal administration may also be suitable, depending on the specific medical use of purified Fc-fusion protein.

For this purpose, in a preferred embodiment of the present invention, the purified Fc-fusion protein may be formulated into pharmaceutical composition, i.e. together with a pharmaceutically acceptable carrier, excipients or the like.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, intracranial, epidural, topical, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The therapeutically effective amounts of the active protein(s) will be a function of many variables, including the type of Fc-fusion protein, the affinity of the Fc-fusion protein for its ligand, the route of administration, the clinical condition of the patient.

A “therapeutically effective amount” is such that when administered, the Fc-fusion protein results in inhibition of its ligand of the therapeutic moiety of the Fc-fusion protein, as explained above and referring particularly to Table 5 above.

The dosage administered, as single or multiple doses, to an individual will vary depending upon a variety of factors, including pharmacokinetic properties of the Fc-fusion protein, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of its ligand of the therapeutic moiety in an individual.

Purified Fc-fusion protein may be used in an amount of 0.001 to 100 mg/kg or 0.01 to 10 mg/kg or body weight, or 0.1 to 5 mg/kg of body weight or 1 to 3 mg/kg of body weight or 2 mg/kg of body weight.

In further preferred embodiments, the purified Fc-fusion protein is administered daily or every other day or three times per week or once per week.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

The present invention also relates to a purified Fc-fusion protein composition comprising an extracellular portion of a member of the tumor necrosis factor receptor (TNFR) superfamily obtained by a method according to the invention as described in detail above, wherein said composition comprises less than 2% or less than 1.5% or less than 1% or less than 0.7% or less than 0.6% or preferably less than 0.5 of protein aggregates. The composition of the invention preferably comprises fully intact Fc-fusion protein that is not missing more than 1 or 2 amino acids at its N- or C-terminus, and more preferably it is not missing any amino acid at its N- or C-terminus.

The present invention further relates to a purified Fc-fusion protein composition comprising an extracellular portion of a member of the tumor necrosis factor receptor (TNFR) superfamily obtained by a method according to the invention, wherein said composition comprises less than 1% or less than 0.8% or less than 0.5% or less than 0.1% of free Fc as defined above.

Such an Fc-fusion protein may e.g. be derived from OX40, a member of the TNFR superfamily. Such OX40-function proteins, e.g. OX40-IgG₁ and OX40-hIG₄mut, may preferably be used for treatment and/or prevention of inflammatory and autoimmune diseases such as Crohn's Disease.

The Fc-fusion protein comprising a therapeutic moiety is preferably selected from an extracellular domain of TNFR1, TNFR2, or a TNF binding fragment thereof.

In a preferred embodiment, such Fc-fusion protein is Etanercept, an Fc-fusion protein containing the soluble part of the p75 TNFR (e.g. WO91/03553, WO 94/06476). Etanercept purified according to the invention may be used e.g. for treatment and/or prevention of Endometriosis, Hepatitis C virus infection, HIV infection, Psoriatic arthritis, Psoriasis, Rheumatoid arthritis, Asthma, Ankylosing spondylitis, Cardiac failure, Graft versus host disease, Pulmonary fibrosis, Crohns disease. Lenercept is a fusion protein containing extracellular components of human p55 TNF receptor and the Fc portion of human IgG, and is intended for the potential treatment of severe sepsis and multiple sclerosis.

In a further preferred embodiment, the Fc-fusion protein comprises a therapeutic moiety selected from an extracellular domain of BAFF-R, BCMA, or TACI, or a fragment thereof binding at least one of Blys or APRIL.

An Fc-fusion protein derived from the BAFF-R, purified in accordance with the present invention, may preferably be used for treatment and/or prevention of autoimmune diseases such as rheumatoid arthritis (RA) and systemic lupus erythematosus (SLE).

A BCMA-Ig fusion protein, purified in accordance with the present invention, may preferably be used for treatment and/or prevention of autoimmune diseases.

An Fc-fusion protein derived from TACI (TACI-Fc) preferably comprises a polypeptide selected from:
a. amino acids 34 to 66 of SEQ ID NO: 2;
b. amino acids 71 to 104 of SEQ ID NO: 2;
c. amino acids 34 to 104 of SEQ ID NO: 2;
d. amino acids 30 to 110 of SEQ ID NO: 2;
e. SEQ ID NO: 3;
f. SEQ ID NO: 4;
g. a polypeptide encoded by a polynucleotide hybridizing to the complement of SEQ ID NO: 5 or 6 or 7 under highly stringent conditions; and
h. a mutein of any of (c), (d), (e), or (f) having at least 80% or 85% or 90% or 95% sequence identity to the polypeptide of (c), (d), (e) or (f);

wherein the polypeptide binds to at least one of Blys or APRIL.

Purified TACI-Fc may preferably be used for preparation of a medicament for treatment and/or prevention of a number of diseases or disorders. Such diseases or disorders are preferably selected from autoimmune disorders such as systemic lupus erythematosus (SLE), rheumatoid arthritis (RA), as well as for treatment of multiple sclerosis (MS). Purified TACI-Fc may also be used for treatment of cancer, such as hematological malignancies such as multiple myeloma (MM) and/or non-Hodgkin's lymphoma (NHL), chronic lymphocytic leukemia (CLL) and Waldenstrom's macroglobulemia (WM).

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Purification of Recombinant, Human TACI-Fc from Serum-Free CHO Cell Supernatant

### Glossary

BV: bed volume
CHO: Chinese Hamster Ovary
DSP: Downstream Process
EDTA: Ethylene Diamine Tetraacetic Acid
ELISA: Enzyme-Linked ImmunoSorbent Assay
HAC: Hydroxyapatite Chromatography
HCP: Host Cell Protein
HPLC: High Performance Liquid Chromatography
id: internal diameter
K: potassium
kD: kilo Dalton
MES: 2-Morpholinoethanesulfonic acid
Na: sodium
NaAc: Sodium Acetate
n/d: not determined
PA-SE-HPLC: Protein A Size-Exclusion High Performance Liquid Chromatography
Ppm: parts per million
RO: Reverse Osmosis
RT: Room Temperature
SDS-PAGE: Sodium Dodecyl Sulphate Polyacrylamide Gel Electrophoresis
SE-HPLC: Size-Exclusion High Performance Liquid Chromatography
T° C.: Temperature
TMAC: Tetra-Methyl Ammonium Chloride
UV: Ultra-Violet
WFI: Water For Injection
WRO Water Reverse Osmosis

### Example 1

### Capture Step: Affinity Purification on Protein A

Starting material was clarified harvest of a TACI-Fc expressing CHO cell clone cultured under serum-free conditions and stored frozen until use.

The Capture Step on a MabSelect Xtra™ column (GE Healthcare 17-5269-03) was carried out according to the following protocol, on a column having a bed height of 17 cm. All operations were performed at room temperature, except for the load solution, which was kept at a temperature below 15° C. The UV signal at 280 nm was recorded.

Sanitization

The column was sanitised with at least 3 BV of 0.1M acetic acid+20% ethanol in reverse flow at 250 cm/h. The flow was stopped for 1 hour.

Wash Step

The column was washed with at least 2 BV of RO water in reverse flow at 250 cm/h.

Equilibration

The column was equilibrated with at least 5 BV of 25 mM sodium phosphate +150 mM NaCl pH7.0 (until conductivity and pH parameters are within specified range: pH 7.0±0.1, conductivity 18±2 mS/cm) in down flow at 450 cm/h.

Loading

The column was loaded with clarified harvest kept at a temperature below 15° C. to a capacity of up to 15 mg total TACI-Fc as determined by Biacore assay per ml of packed resin at a flow rate of 350 cm/h.

Wash Step

Wash the column with at least 2 BV of equilibration buffer at 350 cm/h then with at least 4 BV of equilibration buffer (until the UV signal is back to baseline) at 450 cm/h.

Elution

The material was eluted with different elution buffers as shown in Table I at a flow rate of 350 cm/h. The eluate fraction was collected from start of UV signal increase to 6.0±0.5 BV of elution. The eluate was incubated for 1 hour at room temperature at a pH below 4.1 (adjusted by addition of citric acid solution, if necessary) and then the pH was adjusted to 5.0±0.1 by addition of 32% NaOH solution.

Regeneration

The column was regenerated with at least 3 BV of 50 mM NaOH+1M NaCl in reverse flow at 450 cm/h, stop the flow for 15 min then re-start the flow at 450 cm/h for at least 3 BV (until the UV signal is back to baseline).

From this step, the column was operated in reverse flow mode.

Wash Step

The column was washed with at least 2 BV of RO water at 450 cm/h.

Sanitisation

The column was santitised with at least 3 BV of sanitisation buffer at 250 cm/h, the flow stopped and the column incubated for 60 min.

Final Wash Steps

The column was washed with at least 1 BV of RO water at 250 cm/h, then with at least 3 BV of equilibration buffer at 250 cm/h and finally with at least 2 BV of RO water at 250 cm/h.

Finally, the column was stored after flushing with at least 3 BV of 20% ethanol at 250 cm/h.

Results

TABLE I
Results using different elution buffers

| | | TACI-Fc | | |
|---|---|---|---|---|
| Run | | yield | Aggregates | HCPs |
| # | Elution buffer | (%) | (%) | (ppm) |
| 1 | 50 mM NaAc pH 3.7 | 47.7 | 30.3 | 5558 |
| 2 | 100 mM NaAc pH 3.8 | 55.7 | 25.2 | n/d |
| 3 | 200 mM NaAc pH 3.8 | 58.0 | 28.2 | n/d |
| 4 | 100 mM NaAc pH 3.7 | 68 | 30.0 | n/d |
| 5 | 0.2M NaAc + 150 mM NaCl | 75.1 | 3.8 | n/d |
| | pH 4 | | | |
| 6 | 100 mM NaAc pH 3.7 | 84.6 | 22 | 3491 |
| 7 | 250 mM NaAc pH 3.7 | 82.8 | 18.7 | 3318 |
| 8 | 100 mM Na citrate pH 3.7 | 79.2 | 8.8 | 4710 |
| 9 | 250 mM Na citrate pH 3.7 | 71.9 | 23 | 2347 |
| 10 | 100 mM Na citrate pH 3.75 | 82.8 | 8.5 | 1576 |
| 11 | 100 mM Na citrate pH 3.75 | 66.6 | 9.0 | 664 |
| 12 | 100 mM NaAc pH 3.85 | 83.3 | 15.0 | n/d |
| 13 | 100 mM Na citrate pH 3.75 | 81.0 | 9.1 | 3490 |
| 14 | 100 mM Na citrate pH 3.65 | 75.1 | 14.6 | 2580 |
| 14 | 100 mM Na citrate pH 3.75 | 44.7 | 18.4 | 3783 |
| 16 | 100 mM Na citrate pH 3.75 | 47.1 | 15.8 | 3217 |
| 17 | 100 mM Na citrate pH 3.75 | 50.7 | 9.4 | 2349 |
| 18 | 100 mM Na citrate pH 3.75 | 58.0 | 10.4 | 2550 |
| 19 | 100 mM Na citrate pH 3.75 | 67.1 | 28.7 | 2372 |
| 20 | 100 mM Na citrate pH 3.75 | 65.6 | 17.5 | 2353 |
| 21 | 100 mM Na citrate pH 3.75 | 75.6 | 19.4 | 1807 |
| 22 | 100 mM Na citrate pH 3.75 | 57.1 | 20.7 | 2465 |
| 23 | 100 mM Na citrate pH 3.75 | 51.9 | 18.4 | 2030 |
| 24 | 100 mM Na citrate pH 3.75 | 58 | 11.5 | 1746 |
| 25 | 100 mM Na citrate pH 3.75 | 41.8 | 22.9 | 3029 |
| 26 | 100 mM Na citrate pH 3.9 | 39.4 | 6.0 | 2424 |
| 27 | 100 mM Na citrate pH 3.9 | 31.0 | 8.8 | 2936 |
| 28 | 100 mM Na Ac pH 4.1 | 28.3 | 25.0 | 3311 |
| 29 | 100 mM Na citrate pH 3.9 | 46.4 | 9.1 | n/d |
| 30 | 100 mM NaAc pH 4.1 | 42.8 | 13.4 | n/d |
| 31 | 100 mM Na citrate pH 3.75 | 57.5 | 26.5 | n/d |
| 32 | 100 mM NaAc pH 4.2 | 38.1 | 10.1 | n/d |
| 33 | 100 mM Na citrate pH 3.9 | 43.3 | 8.3 | 2011 |
| 34 | 100 mM Na citrate pH 3.9 | 63.6 | 6.6 | 1749 |
| 35 | 100 mM Na citrate pH 3.9 | 65.7 | 7.3 | 1689 |
| 36 | 100 mM Na citrate pH 3.9 | 62.7 | 7.4 | 1609 |
| 37 | 100 mM Na citrate pH 3.9 | 61.6 | 7.4 | 1479 |
| 38 | 100 mM Na citrate pH 3.9 | 60.6 | 7.4 | 1623 |
| 39 | 100 mM Na citrate pH 3.9 | 64.6 | 8.0 | 1497 |

Conclusions

TACI-Fc5 in clarified harvest was captured directly on a MabSelect Xtra column at a dynamic capacity of 15 g total TACI-Fc5 per L of packed resin at a flow rate of 350 cm/h. Elution conditions, especially pH, were optimized to maximize recovery of product while providing significant reduction in aggregate levels. An elution buffer of 0.1 M sodium citrate pH 3.9 was selected giving about 5-10% aggregate levels starting from about 25-40% in clarified harvest and with no turbidity observed. HCP levels were typically 1500-2000 ppm. The HCP levels were measured by ELISA using polyclonal antibodies. The antibody mixture was generated against host cell proteins derived from clarified and concentrated cell culture supernatant of non-transfected CHO cells.

### Example 2

### Cation Exchange Chromatography

The eluate from the capture step on Protein A, dialysed into suitable loading buffer, was used as a starting material for the cation exchange chromatography.

A Fractogel EMD SO₃⁻ column (Merck 1.16882.0010) having a bed height of 10 cm was used in this step. A Fractogel SO₃⁻ column with a bed height of 15 cm may be used as well. In the latter case, the dynamic capacity and flow rate may need adaptation, which is well within routine knowledge of the person skilled in the art.

All the operations were performed at room temperature and the flow rate was kept constant at 150 cm/h. The UV signal at 280 nm was recorded at all time.

Wash Step

The column was washed with at least 1 BV of WRO (water reverse osmosis).

Sanitisation

Then, the column was sanitised with at least 3 BV of 0.5M NaOH+1.5M NaCl in up-flow mode.

Rinsing

The column was rinsed with at least 4 BV of WRO in down-flow mode.

Equilibration

The column was equilibrated with at least 4 BV of 100 mM sodium citrate pH5.0 (or until the target conductivity of 12±1 mS/cm and pH 5.0±0.1 are reached).

Loading

The column was loaded with post capture material at pH 5.0 (pH at 5.0±0.1, conductivity at 12±1 mS/cm) and at a capacity of no more than 50 mg TACI-Fc, as determined by SE-HPLC assay per ml of packed resin.

Wash Step

The column was then washed with at least 5 BV of 100 mM sodium phosphate pH6.5.

Elution

The column was eluted with different buffers and under different conditions as reported in tables II-IV below.

Regeneration and Sanitisation

The column was regenerated and sanitised with 4 BV of 0.5M NaOH+1.5M NaCl in up-flow mode. Then, the flow was stopped for 30 min. Rinsing

The column was rinsed with at least 4 BV of WRO.

Storing

The column was stored in at least 3 BV of 20% ethanol.

Results

TABLE II
Effect of elution pH and conductivity
HCP levels in the load: 189 ppm

| | Conductivity | | | HCP |
|---|---|---|---|---|
| pH | (mS/cm) | TACI-Fc recovery | HCPs (ppm) | clearance (x) |
| 6.5 | 15.0 | 25% | 118 | 1.6 |
| 7.3 | 22.5 | 100% | 50 | 3.8 |
| 8.0 | 15.0 | 95% | 34 | 5.5 |
| 7.3 | 22.5 | 100% | 56 | 3.4 |
| 7.3 | 33.0 | 98% | 133 | 1.4 |
| 7.3 | 22.5 | 96% | 45 | 4.2 |
| 7.3 | 22.5 | 97% | 53 | 3.6 |
| 7.3 | 12.0 | 54% | 79 | 2.4 |
| 6.3 | 22.5 | 83% | 47 | 4.1 |
| 8.0 | 30.0 | 96% | 108 | 1.8 |
| 8.2 | 22.5 | 97% | 46 | 4.2 |
| 6.5 | 30.0 | 91% | 116 | 1.6 |
| 7.3 | 22.5 | 93% | 48 | 3.9 |
| 7.3 | 22.5 | 95% | 40 | 4.8 |

Table III shows the TACI-Fc recovery and HCP clearance when loading at a capacity of 10 and 32 mg TACI-Fc per ml of resin and eluting in a phosphate buffer at a conductivity of between 12 to 33 mS/cm. Collection of the peak was done from the beginning of the UV increase for 10±0.5 BV.

TABLE III
Effect of optimal elution pH and conductivity when loading at capacity
HCP levels in load: 201 ppm

| Loading | | | | | HCP |
|---|---|---|---|---|---|
| capacity | | Conductivity | TACI-Fc | HCPs | clearance |
| (mg/ml) | pH | (mS/cm) | recovery | (ppm) | (x) |
| 10 | 8.0 | 15.0 | 91% | 67 | 3.0 |
| | | 20.7 | 93% | 61 | 3.3 |
| 32 | 8.0 | 20.7 | 88% | 54 | 3.7 |

Table IV shows the effect of a wash step with 50 or 100 or 150 mM sodium phosphate pH 6.5 on TACI-Fc recovery and HCP clearance.

TABLE IV
Effect of wash step conditions on column performance
HCP levels in the load: 190 ppm and aggregate levels: 2.0%

| Phosphate | | TACI-Fc | TACI-Fc | | HCPs in |
|---|---|---|---|---|---|
| concentration | | yield in | yield in | Aggregates | eluate |
| in wash (mM) | | wash | eluate | in eluate | (ppm) |
| wash 1 | 50 | 0.7% | 99% | 2.8% | 62 |
| wash 2 | 100 | 2.1% | 98% | 2.9% | 59 |
| wash 3 | 150 | 9.1% | 90% | 2.7% | 49 |

The buffer used in wash 2, containing 100 mM sodium phosphate pH 6.5, had a conductivity of 8.4 mS/cm.

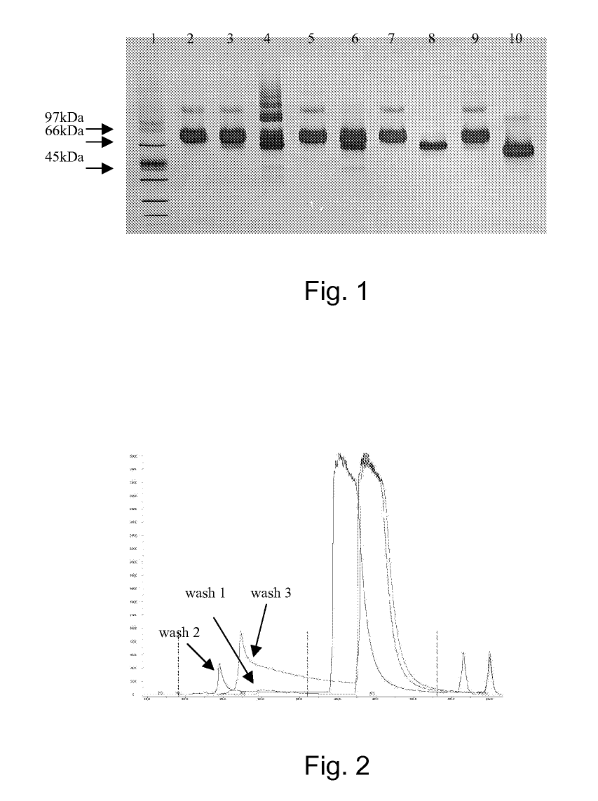

FIG. 1 shows a silver stained, non-reduced SDS-PAGE gel of samples derived from experiments using the three wash step conditions shown in Table IV on the free Fc clearance.

FIG. 2 shows overlapping chromatograms of the wash step experiments with sodium phosphate at different concentrations.

The wash step was optimized at pH 6.5 with increasing concentrations of sodium phosphate (50 to 150 mM). As can be seen in FIG. 1, a wash buffer concentration of 150 mM (wash 3, lane 6) resulted in losses of TACI-Fc. A wash buffer concentration of 50 mM (wash 1, lane 8) resulted in a peak of pure TACI-Fc, however, the eluate contained traces of free Fc. A wash step with 100 mM sodium phosphate pH 6.5 resulted in 98% recovery in the main peak of elution and only 2% losses in the wash (FIG. 2). HCP clearance was 3.2 fold. Analysis of wash and eluate fractions by SDS-PAGE show that the wash step contained Free Fc with some intact TACI-Fc at buffer concentrations of 100 mM or above (FIG. 1, lanes 4 and 6). A concentration of 100 mM or more is necessary to completely remove Free Fc from the eluate fraction (FIG. 1, lanes 5 and 7).

Conclusions

A cation-exchange step was developed as a second purification step, after the capture step. The capture eluate was at low pH (5.0) and low conductivity and could be directly loaded onto the cation-exchanger. A Fractogel EMD SO₃⁻ resin was selected with a loading capacity of 50 mg/ml. The non-bioactive degradation product free Fc could be efficiently removed in a wash step with 0.1 M sodium phosphate pH 6.5. Elution conditions were optimised for best clearance of HCPs and high TACI-Fc recovery (179 mM sodium phosphate pH 8.0, conductivity 20.7 mS/cm).

Alternatively, elution can be carried out in 10 BV of 20 mM sodium phosphate and 180 mM NaCl pH8.0 from the start of the rise in absorbance at 280 nm.

### Example 3

### Anion Exchange Chromatography

The starting material used for this purification step was the eluate from the cation exchange step on Fractogel SO₃⁻ (see Example 2), dialysed or diluted into suitable loading buffer.

This anion-exchange chromatography step was carried out on a SOURCE 30Q column (GE Healthcare 17-1275-01) with a bed height of 10 cm. A SOURCE 30Q column with a bed height of 15 cm may be used as well in this step. In the latter case, the dynamic capacity and flow rate may need adaptation, which is well within routine knowledge of the person skilled in the art.

All operations were carried out at room temperature and the UV signal at 280 nm was recorded. The steps were carried out at a flow rate of either 150 or 200 cm/h.

Rinsing

First, the column was rinsed with at least 1 BV of RO water at a flow rate of 150 cm/h.

Sanitisation

Then, the column was sanitised with at least 3 BV of 0.5M NaOH+1.5M NaCl.

Wash Step

The column was washed with at least 3 BV, preferably 4 to 10 BV of 0.5M Na phosphate pH 7.5 at a flow rate of 200 cm/h.

Equilibration

The column was equilibrated with at least 5 BV of 10, 15, 20, 25, or 30 mM sodium phosphate pH 7.5. Optionally, the column can be pre-equilibrated with 3 BV of 0.5M sodium phosphate pH7.5.

Loading, Washing and Concomitant Collection of TACI-Fc in the Flow-Through

The column was loaded with post-cation exchange material diluted to obtain a phosphate concentration of 10 to 30 mM, pH 7.5, at a capacity of no more than 50 mg TACI-Fc as determined by SE-HPLC assay per ml of packed resin, collecting the flow-through from start of UV increase until the end of the wash step, which is carried out in 4±0.5 BV of equilibration buffer.

Regeneration/Sanitisation

The column was regenerated and sanitised with at least 3 BV of 0.5M NaOH+1.5M NaCl in reverse flow mode (until UV signal is back to the baseline) at a flow rate of 150 cm/h. At the end of the regeneration, the pump is stopped for 30 min.

Wash Step

The column was washed with at least 3 BV of RO water at a flow rate of 200 cm/h.

Storing

The column is stored in at least 3 BV of 20% ethanol (v/v) at a flow rate of 150 cm/h.

Results

The following Table V summarizes the results obtained with the purification process described above.

TABLE V
Effect of loading phosphate concentation

| | | Load | | | | |
|---|---|---|---|---|---|---|
| | Load | TACI-Fc | Load | | | |
| Load | phosphate | conc | capacity | TACI-Fc | Aggre- | HCPs |
| pH | conc (mM) | (mg/L) | (mg/ml) | recovery | gates | (ppm) |
| 7.5 | 30 | 773 | 39 | 94% | 10.4% | 82.8 |
| 7.5 | 25 | 639 | 39 | 90% | 6.9% | 50.4 |
| 7.5 | 20 | 651 | 49 | 90% | 5.6% | 43.9 |
| 7.5 | 15 | 437 | 46 | 88% | 3.4% | 45.0 |
| 7.5 | 10 | 283 | n./d. | 82% | 2.8% | 26.3 |

Conclusions

The anion-exchange step on a Source 30Q column in flow-through mode was optimised to maximise clearance of HCPs and aggregates. Loading cation-exchange eluate either diluted or diafiltered in 20 mM sodium phosphate buffer at pH7.5 gave the best compromise between product recovery (90%) and clearance of HCPs (from about 2000 ppm to 44 ppm) and aggregates (from about 25% to 5.6%). Dynamic capacity of 50 mg TACI-Fc per ml of packed resin at a flow rate of 150-200 cm/h was used.

### Example 4

### Hydroxyapatite Chromatography

The starting material used for this purification step was anion-exchange chromatography flow-through (see Example 3).

A CHT Ceramic Hydroxyapatite Type I, 40 μm column (Biorad 157-0040) with a bed height of 10 cm was used.

All operations were carried out at room temperature. The flow rate was kept constant at 175 cm/h and the UV signal at 280 nm was recorded. All solutions were sterile filtered and the equipment sanitised with sodium hydroxide before use. The column was stored in 0.5M NaOH solution when not in use.

Initial Wash Steps (Rinsing and Pre-Equilibration)

The column was washed with at least 1 BV of 20 mM sodium phosphate pH7.5 buffer, and then with at least 3 BV of 0.5M sodium phosphate buffer pH7.5 to lower the pH.

Equilibration

The column was equilibrated with at least 5 BV of 20 mM sodium phosphate pH7.5 (or until the target conductivity of 3.0±0.3 mS/cm and pH 7.5±0.1 were reached).

Loading

The column was loaded with the SOURCE 30Q flow-through with calcium chloride added to 0.1 mM final concentration from a stock solution at 0.5M and pH adjusted to 7.0 by addition of 85% ortho-phosphoric acid, at a capacity of NMT 50 mg TACI-Fc as determined by SE-HPLC assay per ml of packed resin. It is also possible to load the SOURCE 30Q flow-through without calcium chloride, adjusted to pH 7.0, on the hydroxyapatite column.

Wash Steps

The column was washed with at least 4 BV of 3, 4 or 5 mM sodium phosphate, 10 mM MES, 0.1 mM CaCl₂ pH6.5. In these steps, it is also possible to use the same buffer without calcium chloride.

Elution

The column was eluted with 5, 4, 3 or 2 mM sodium phosphate (see Table VI), 10 mM MES, 0.1 mM CaCl₂, and 0.6, 0.7, 0.8 or 0.9 M KCl pH 6.5 buffer (see Table VII) from the beginning of the UV increase for different BV (see Tables VI and VII). It is also possible to use the same buffer without calcium chloride for the elution.

Rinsing

The column was rinsed with:
at least 1 BV of 20 mM sodium phosphate pH7.5 buffer;
at least 3 BV of 0.5M sodium phosphate pH7.5 buffer; and
with at least 1 BV of 20 mM sodium phosphate pH7.5 buffer.

Storing

The column was stored in at least 3 BV of 0.5M NaOH.

Results

Table VI shows the effect of phosphate concentration (from 2 to 5 mM) in the elution buffer on the clearance of aggregates and product recovery. Elution peak fractions were pooled and analysed by SE-HPLC for TACI-Fc concentration and aggregate levels.

TABLE VI
Effect of phosphate concentration in the elution buffer

| Phosphate conc | | | |
|---|---|---|---|
| (mM) | BV of elution | TACI-Fc yield | Aggregates |
| 5 | 12 | 73% | 0.49% |
| | 13 | 74% | 0.52% |
| | 14 | 68% | 0.65% |
| | 15 | 77% | 0.67% |
| | 16 | 77% | 0.70% |
| | 17 | 70% | 0.73% |
| | 18 | 76% | 0.85% |
| 4 | 12 | 68% | 0.34% |
| | 13 | 67% | 0.29% |
| | 14 | 66% | 0.36% |
| | 15 | 67% | 0.39% |
| | 16 | 66% | 0.38% |
| | 17 | 66% | 0.32% |
| | 18 | 66% | 0.40% |
| 3 | 12 | 70% | 0.46% |
| | 13 | 76% | 0.42% |
| | 14 | 73% | 0.51% |
| | 15 | 71% | 0.52% |
| | 16 | 69% | 0.55% |
| | 17 | 69% | 0.50% |
| | 18 | 70% | 0.53% |
| 2 | 12 | 65% | 0.19% |
| | 13 | 66% | 0.00% |
| | 14 | 66% | 0.18% |
| | 15 | 68% | 0.14% |
| | 16 | 66% | 0.17% |
| | 17 | 71% | 0.19% |
| | 18 | 65% | 0.16% |

Table VII shows the effect of KCl concentration in the elution buffer on the clearance of aggregates and product recovery. Two sodium phosphate concentrations were investigated: 2 and 3 mM. Elution peak fractions were pooled and analysed by SE-HPLC for TACI-Fc concentration and aggregate levels.

TABLE VII
Effect of potassium chloride concentration in the elution buffer

| Phosphate conc | | BV of | TACI-Fc | |
|---|---|---|---|---|
| (mM) | KCl conc (M) | elution | yield | aggregates |
| 3 | 0.6 | 10 | 102% | 0.48% |
| | | 11 | 109% | 0.46% |
| | | 12 | 106% | 0.43% |
| | | 13 | 105% | 0.42% |
| | | 14 | 103% | 0.43% |
| 3 | 0.7 | 10 | 96% | 0.42% |
| | | 11 | 97% | 0.40% |
| | | 12 | 98% | 0.41% |
| | | 13 | 96% | 0.40% |
| | | 14 | 96% | 0.43% |
| 3 | 0.8 | 10 | 106% | 0.58% |
| | | 11 | 110% | 0.55% |
| | | 12 | 112% | 0.57% |
| | | 13 | 101% | 0.59% |
| | | 14 | 110% | 0.57% |
| 2 | 0.6 | 10 | 71% | 0.29% |
| | | 11 | 79% | 0.28% |
| | | 12 | 80% | 0.29% |
| | | 13 | 80% | 0.29% |
| | | 14 | 81% | 0.26% |
| 2 | 0.9 | 10 | 64% | 0.27% |
| | | 11 | 72% | 0.25% |
| | | 12 | 73% | 0.29% |
| | | 13 | 70% | 0.33% |
| | | 14 | 66% | 0.24% |

Conclusions:

Hydroxyapatite chromatography provides a reliable, efficient way of reducing TACI-Fc aggregate levels. Starting from anion-exchange chromatography purified material (see Example 3) with aggregate levels of about 5-8%, hydroxyapatite chromatography can reduce these levels to below 0.8% with a recovery of TACI-Fc of 85-90%.

Overall Result

A four-step purification process for TACI-Fc has been developed resulting in highly purified TACI-Fc with an overall reduction of aggregates to less than 1% (0.2-0.8% in five experiments), overall reduction of HCPs to about 5-10 ppm and an overall reduction of free Fc levels to less than 0.5% (0.2 and 0.1% in two experiments).

### REFERENCES

1. Akerstrom and Bjork, J Biol Chem. 1989 Nov. 25; 264(33):19740-6.
2. Altschul S F et al, J Mol Biol, 215, 403-410, 1990
3. Altschul S F et al, Nucleic Acids Res., 25:389-3402, 1997
4. Armour K L. et al., 1999. Recombinant human IgG molecules lacking Fcgamma receptor I binding and monocyte triggering activities. Eur J Immunol. 29(8):2613-24
5. Black et al., US 2002/0115175
6. Bodmer et al., TIBS 27(1), 19-24
7. Boschetti, E., Jungbauer, Sep. Sci. & Tech. 2 No. 15, Acad. Press (2000) 53
8. Boschetti et al., Genetic Engineering Vol. 20, No. 13, July, 2000
9. Bossen et al., JBC 281(2), 13964-13971
10. Bram and von Bulow, U.S. Pat. No. 5,969,102 (1999)
11. Bram et al., WO 98/39361
12. Carter P J., 2006. Potent antibody therapeutics by design. Nature Reviews Immunology. Advance online publication
13. Devereux J et al, Nucleic Acids Res, 12, 387-395, 1984
14. Feng et al., Bioprocessing 2005, 1-7
15. Giovannini, Biotechnology and Bioengineering 73:522-529 (2000)
16. Grantham et al., Science, Vol. 185, pp. 862-864 (1974)
17. Gross et al., Nature 404(27), 995-999
18. Gross et al., WO 00/40716
19. Hinton P R. et al., 2004. Engineered human IgG antibodies with longer serum half-lives in primates. J Biol Chem. 279(8):6213-6
20. Hymowitz et al., JBC 280(8), 7218-7227
21. Idusogie E E. et al., 2000. Mapping of the C1q binding site on rituxan, a chimeric antibody with a human IgG1Fc. J. Immunol. 164(8):4178-84
22. Idusogie E E. et al., 2001. Engineered antibodies with increased activity to recruit complement. J Immunol. 166(4):2571-5
23. Ishihara, EP 1 614 693
24. Kabat, E. A., Wu, T. T., Perry, H. M., Gottesman, K. S., and Foeller, C. (1991), Sequences of Proteins of Immunological Interest, 5th Ed., National Institutes of Health, Bethesda, Md.
25. Kochanek et al., EP 1 230 354
26. Locksley et al., Cell 104, 487-501, 2001
27. Melchers, Ann. Rheum. Dis 2003, 62, 25-27.
28. Moore et al., Science 285: 260-3
29. Moreau et al., Blood 103(8), 3148-3157
30. Naismith and Sprang, TIBS 23, 74-79, 1998
31. Novak et al., Blood 103(2), 689-694
32. Rixon et al., WO 02/094852
33. Pearson, Methods Enzymol. 1990; 183:63-98
34. Porath, J. Carlsson, I. Olsson, and G. Belfrage, Nature (London) 258, 598-599 (1975)
35. Porath and B. Olin, Biochemistry 22, 1621-1630 (1983)
36. Puren et al., Proc Natl Acad Sci USA. 1999 Mar. 2; 96(5):2256-61
37. Shepard, J. of Chromatography 891:93-98 (2000)
38. Shields R L. et al., 2001. High resolution mapping of the binding site on human IgG1 for Fc gamma RI, Fc gamma RII, Fc gamma RIII, and FcRn and design of IgG1 variants with improved binding to the Fc gamma R. J Biol Chem. 276(9):6591-604.
39. Smith et al., WO 91/03553
40. Smith et al., WO 94/06476
41. Stanker, J. Immunological Methods 76:157-169 (1985) (10 mM to 30 mM sodium phosphate elusion gradient)
42. Steurer W. et al., 1995. Ex vivo coating of islet cell allografts with murine CTLA4/Fc promotes graft tolerance. J Immunol. 155(3):1165-74
43. Sun et al., WO 2005/044856
44. Takeda et al., EP 1 561 756
45. Tarditi, J. Chromatography 599:13-20 (1992)
46. Vaccaro C. et al., 2005. Engineering the Fc region of immunoglobulin G to modulate in vivo antibody levels. Nat Biotechnol. 23(10):1283-8
47. Vigers et al., Nature. 1997 Mar. 13; 386(6621):190-4
48. Vedantham et al., WO 03/59935
49. Vola et al., BioTechniques 14:650-655 (1993)
50. von Bulow and Bram, Science 228: 138 (1997)
51. Xia et al., J. Exp. Med. 2000, 137-143.

1. A method for purifying an Fc-fusion protein having an isoelectric point (pI) from 6.9 to 9.5, comprising:
a. subjecting a fluid comprising said Fc-fusion protein to Protein A or Protein G affinity chromatography to obtain a first eluate;
b. subjecting the first eluate of step (a) to cation exchange chromatography to obtain a second eluate;
c. subjecting the second eluate of step (b) to anion exchange chromatography to obtain a flow-through;
d. subjecting the flow-through of step (c) to hydroxyapatite chromatography to obtain a third eluate comprising purified Fc-fusion protein.

2. The method according to claim 1, wherein elution in step (a) is carried out at a pH ranging from 2.8 to 4.5.

3. The method according to claim 1, wherein step (b) further comprises:
b.1. washing the cation exchange resin after loading with a buffer having a pH ranging from 6 to 7 and a conductivity ranging from 6 to 10 mS/cm; and
b.2. eluting the column at a pH ranging from 7.3 to 8.2 and a conductivity ranging from 15 to 22 mS/cm.

4. The method according to claim 1, wherein in step (c), equilibration and loading is carried out in a buffer having a conductivity of 3 to 4.6 mS/cm and a pH of one unit below the pI value of the Fc-fusion protein.

5. The method according to claim 1, wherein elution in step (d) is carried out in the presence of sodium phosphate at a concentration ranging from 3 to 10 mM.

6. The method according to claim 1, wherein elution in step (d) is carried out in the presence of potassium chloride ranging from 0.4 to 1 M.

7. The method according to claim 1, wherein the elution in step (d) is carried out at a pH ranging from 6 to 7.

8. The method according to claim 1 or claim 2, wherein step (a) is carried out on a resin comprising cross-linked agarose modified with recombinant Protein A or Protein G.

9. The method according to claim 1 or claim 3, wherein step (b) is carried out on a strong cation exchange resin.

10. The method according to claim 9, wherein said resin comprises a cross-linked methacrylate modified with SO₃⁻ groups.

11. The method according to claim 1 or claim 4, wherein step (c) is carried out on a strong anion exchange resin.

12. The method according to claim 11, wherein said resin comprises polystyrene/divinyl benzene modified with N⁺(CH₃)₃.

13. The method according to any one of claims 1, 5, 6 and 7, wherein step (d) is carried out on a ceramic hydroxyapatite resin.

14. The method according to claim 13, wherein the ceramic hydroxyapatite resin comprises particles having a size of 40 p.m.

15. The method according to claim 1, further comprising at least one step of ultrafiltration.

16. The method according to claim 15, wherein the ultrafiltration step is carried out between steps (b) and (c) and/or after step (d).

17. The method according to claim 1, further comprising formulating the Fc-fusion protein into a pharmaceutical composition.

18. The method according to claim 1 or claim 17, wherein the Fc-fusion protein has a pI between 8 and 9.

19. The method according to claim 18, wherein the Fc-fusion protein has a pI between 8.3 and 8.6.

20. The method according to claim 1 or claim 17, wherein the Fc-fusion protein comprises a ligand binding portion of a member of the tumor necrosis factor receptor (TNFR) superfamily.

21. The method according to claim 20, wherein the ligand binding portion is selected from an extracellular domain of TNFR1, TNFR2, or a TNF binding fragment thereof.

22. The method according to claim 20, wherein the ligand binding portion selected from an extracellular domain of BAFF-R, BCMA, TACI, or a fragment thereof binding at least one of Blys or APRIL.

23. The method according claim 22, wherein the Fc-fusion protein comprises a polypeptide selected from
(a) amino acids 34 to 66 of SEQ ID NO: 2;
(b) amino acids 71 to 104 of SEQ ID NO: 2;
(c) amino acids 34 to 104 of SEQ ID NO: 2;
(d) amino acids 30 to 110 of SEQ ID NO: 2;
(e) SEQ ID NO: 3;
(f) SEQ ID NO: 4;
(g) a polypeptide encoded by a polynucleotide hybridizing to the complement of SEQ ID NO: 5 or 6 or 7 under highly stringent conditions; and
(e) a mutein of any of (c), (d), (e), or (f) having at least 80% sequence identity to the polypeptide of (c), (d), (e) or (f);
wherein the polypeptide binds to at least one of Blys or APRIL.

24. The method according to claim 1, wherein the Fc-fusion protein comprises a heavy chain constant region of an immunoglobulin.

25. The method according to claim 24, wherein the constant region is a human constant region.

26. The method according to claim 24, wherein the immunoglobulin is an IgG₁.

27. The method according to claim 24, wherein the constant region comprises the hinge, CH2 and a CH3 domain.

28. A purified Fc-fusion protein composition obtained by a method according to claim 20, wherein the Fc-fusion protein comprises a polypeptide selected from
a) amino acids 34 to 66 of SEQ ID NO: 2;
(b) amino acids 71 to 104 of SEQ ID NO: 2;
(c) amino acids 34 to 104 of SEQ ID NO: 2;
(d) amino acids 30 to 110 of SEQ ID NO: 2;
(e) SEQ ID NO: 3;
(f) SEQ ID NO: 4;
g) a polypeptide encoded by a polynucleotide hybridizing to the complement of SEQ ID NO: 5 or 6 or 7 under highly stringent conditions; and
(e) a mutein of any of (c), (d), (e) or (f) having at least 80% sequence identity to the polypeptide of (c), (d), (e) or (f);
wherein the polypeptide binds to at least one of Blys or APRIL, and
wherein said composition comprises less than 1% of protein aggregates, and
wherein said composition comprises less than 1% of free Fc protein.

29. The purified Fc-fusion protein composition of claim 28, wherein said composition comprises less than 0.5% of protein aggregates.

30. The purified Fc-fusion protein composition of claim 28, wherein said composition comprises less than 0.5% of free Fc protein.

31. The purified Fc-fusion protein composition of claim 28, wherein said composition comprises less than 0.1% of free Fc protein.

32. The method according to claim 23, wherein the Fc-fusion protein comprises a heavy chain constant region of an immunoglobulin.

33. The method according to claim 32, wherein the constant region is a human constant region.

34. The method according to claim 32, wherein the immunoglobulin is an IgG1.

35. The method according to claim 32, wherein the constant region comprises the hinge, CH2 and a CH3 domain.


SEQUENCE LISTING (7 sequences)

SEQ ID NO: 1 - protein, 40 aa, artificial sequence
  source: /note=description of artificial sequence consensus sequence, in which the specific amino acids at positions 3, 12, 14, 15, 18, 21, 25, 34 and 38 are spaced by any amino acid
XXCXXXXXXXXDXLLXXCXXCXXXCXXXXXXXXCXXXCXX

SEQ ID NO: 2 - protein, 293 aa, homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 3 - protein, 251 aa, artificial sequence
  source: /note=description of artificial sequence this is a portion of a human immunoglobulin heavy chain
MKHLWFFLLLVAAPRWVLSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 348 aa, artificial sequence
  source: /note=description of artificial sequence this is a fusion protein sequence containing a portion of a human TACI receptor and a portion of a human immunoglobulin heavy chain sequence.
MDAMKRGLCCVLLLCGAVFVSLSQEIHAELRRFRRAMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 879 bp, homo sapiens
atgagtggcctgggccggagcaggcgaggtggccggagccgtgtggaccaggaggagcgctttccacagggcctgtggacgggggtggctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggattggagcacagaggctcagaagcaagtccagctctcccggggctgaagctgagtgcagatcaggtggccctggtctacagcacgctggggctctgcctgtgtgccgtcctctgctgcttcctggtggcggtggcctgcttcctcaagaagaggggggatccctgctcctgccagccccgctcaaggccccgtcaaagtccggccaagtcttcccaggatcacgcgatggaagccggcagccctgtgagcacatcccccgagccagtggagacctgcagcttctgcttccctgagtgcagggcgcccacgcaggagagcgcagtcacgcctgggacccccgaccccacttgtgctggaaggtgggggtgccacaccaggaccacagtcctgcagccttgcccacacatcccagacagtggccttggcattgtgtgtgtgcctgcccaggaggggggcccaggtgca

SEQ ID NO: 6 - DNA, 753 bp, artificial sequence
  source: /note=description of artificial sequence DNA encoding SEQ ID NO3
atgaagcacctgtggttcttcctcctgctggtggcggctcccagatgggtcctgtccgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - DNA, 1044 bp, artificial sequence
  source: /note=description of artificial sequence DNA encoding the protein of SEQ ID NO 4
atggatgcaatgaagagagggctctgctgtgtgctgctgctgtgtggcgccgtcttcgtttcgctcagccaggaaatccatgccgagttgagacgcttccgtagagctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa